(19) United States
(12) Patent Application Publication
(10) Pub. No.: US 2003/0059442 A1
(43) Pub. Date: Mar. 27, 2003

(54) Attenuated microorganisms for the treatment of infection

(76) Inventors: Gordon Dougan, Winnersh Triangle (GB); David William Holden, London (GB); Joseph David Santangelo, Winnersh Triangle (GB); Jacqueline Elizabeth Shea, Winnersh Triangle (GB); Francis Richard Brennan, Winnersh Triangle (GB)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/169,047

(22) PCT Filed: Dec. 22, 2000
(86) PCT No.: PCT/GB00/05002

(30) Foreign Application Priority Data
Dec. 23, 1999 (GB) 9930457.8
Dec. 23, 1999 (GB) 9930460.2
Dec. 23, 1999 (GB) 9930455.2
Dec. 23, 1999 (GB) 9930456.0
Dec. 23, 1999 (GB) 9930459.4
Dec. 23, 1999 (GB) 9930458.6
Dec. 23, 1999 (GB) 9930461.0

(51) Int. Cl. A61K 39/02; C12N 1/21

(52) U.S. Cl. 424/200.1; 435/252.3

(57) ABSTRACT

Double mutant Salmonella microorganisms help prevent reactivity of the microorganism while maintaining the effectiveness of the microorganism to elicit an immune response. Various specific combinations of mutants are beneficial.

### FIELD OF THE INVENTION

This invention relates to attenuated microorganisms that can be used in vaccine compositions for the prevention or treatment of bacterial or viral infections.

### BACKGROUND OF THE INVENTION

It is well established that live attenuated micro-organisms are highly effective vaccines; immune responses elicited by such vaccines are often of greater magnitude and of longer duration than those produced by non-replicating immunogens. One explanation for this may be that live attenuated strains establish limited infections in the host and mimic the early stages of natural infection. In addition, unlike killed preparations, live vaccines are able to induce potent cell-mediated responses which may be connected with their ability to replicate in antigen-presenting cells, such as macrophages.

There has been a long history of the use of live attenuated Salmonella vaccines as safe and effective vaccines for the prevention of salmonellosis in animals and humans. Indeed, the live attenuated oral typhoid vaccine, Ty21a (Vivotif), manufactured by the Swiss Serum Vaccine Institute, has proved to be a very successful vaccine for the prevention of typhoid fever and has been licensed in many countries including the US and Europe.

However, the attenuation of this strain was achieved using chemical mutagenesis techniques and the basis of attenuation of the strain is not fully understood. Because of this, the vaccine is not ideal in terms of the number of doses (currently four) and the number of live organisms that have to be given at each dose.

Modern molecular biology techniques, coupled with the increasing knowledge of Salmonella pathogenesis, has led to the identification of several genes that are essential for the in vivo growth and survival of the organisms. This has provided new gene targets for attenuation, leading to the concept that future vaccine strains can be ‘rationally’ attenuated by introducing defined non-reverting mutations into selected genes known to be involved in virulence. This will facilitate the development of improved vaccines, particularly in terms of the immunogenicity and therefore the number of doses that have to be given.

Although many attenuated strains of Salmonella are now known, few have qualified as potential vaccine candidates for use in humans. This may be due in part to the need to balance the immunogenicity of the vaccine with the possibility of the Salmonella microorganism becoming reactive.

It is clear that the selection of appropriate targets for attenuation which will result in a suitable vaccine candidate, is not straightforward and cannot easily be predicted. Many factors may influence the suitability of the attenuated strain as an appropriate vaccine, and there is much research being carried out to identify suitable strains. For example, many attenuated strains tested as vaccine candidates lead to vaccinemia or abscesses in the patient.

It is therefore desirable to develop a vaccine having a high degree of immunogenicity with reduced possibility of the microorganism strain reverting to an reactive form.

### SUMMARY OF THE INVENTION

The present invention is based on the finding that several combinations of attenuating mutations introduced into a Salmonella microorganism can produce a vaccine having a high degree of immunogenicity and a low risk of the microorganism reverting to a reactive form. The resulting vaccine strains exhibit good side-effect profiles.

According to a first aspect of the invention, a Salmonella microorganism has an attenuating mutation which disrupts the expression of a gene located within the Spi2 pathogenicity island, and a further mutation which disrupts the expression of any of the genes clpP, ompR, sifA, sseC or ssaB.

According to a second aspect of the invention, a Salmonella microorganism has an attenuating mutation which disrupts the expression of an aro gene, and a further mutation which disrupts the expression of any of the genes clpP or sifA.

The Salmonella microorganisms may be used in the manufacture of a medicament for intravenous or oral delivery for the treatment of a bacterial or viral infection, e.g. for the treatment of typhoid.

### DESCRIPTION OF THE INVENTION

The microorganisms and vaccine compositions of the present invention may be prepared by known techniques.

The choice of particular Salmonella microorganism and the selection of the appropriate mutation, can be made by the skilled person without undue experimentation. A preferred microorganism is Salmonella typhimurium.

A first set of mutants comprises a first mutation in a gene located within the region of the Salmonella pathogenicity island two (Spi2); this region is disclosed in WO-A-9617951.

Spi2 is one of two classical pathogenicity islands located on the Salmonella chromosome. Spi2 comprises several genes that encode a type III secretion system involved in transporting Spi2-encoded virulence-associated proteins (so-called effector proteins) outside of the Salmonella bacteria and potentially directly into target host cells such as macrophages. Part of Spi2 (the apparatus genes) encodes the secretion apparatus of the type III system. Spi2 is absolutely essential for the pathogenesis and virulence of Salmonella in the mouse, an observation now documented by several different groups around the world. S. typhimurium Spi2 mutants are highly attenuated in mice challenged by the oral, intravenous and intraperitoneal routes of administration.

In a preferred embodiment, the gene in the Spi2 region is an apparatus gene. Apparatus genes located within Spi2 are now well characterised; see for example Hensel et al., Molecular Microbiology, (1997); 24(1): 155-167. Genes suitable for use in the present invention include ssaV, ssaJ, ssaK, ssaL, ssaM, ssaO, ssaP, ssaQ, ssaR, ssaS, ssaT, ssaU and ssaH genes.

The mutation in the Spi2 region does not necessarily have to be within a gene to disrupt the function. For example, a mutation in an upstream regulatory region may also disrupt gene expression, leading to attenuation. Mutations in an intergenic region may also be sufficient to disrupt gene function.

In a preferred embodiment of the invention, the Spi2 gene is ssaV and the further mutation disrupts any of clpP, ompR, sifA or sseC. In a separate preferred embodiment, the mutation disrupts ssaT and the further mutation disrupts ssaB.

The clpP gene is described in Gifford et al., Gen. Microbiol., 1993; 139:913-920. The encoded protein is a stress-response protease.

The ompR gene is described in Chatfield et al., Infection and Immunity, 1991; 59(1): 449-452. The encoded protein is a component of a two-component system (OmpR-EnvZ) with a global regulatory function, and is also a regulator for the two-component system ssrA-ssrB in Spi2 (Lee et al., J. Bacteriol., 2000; 182(3): 771-781).

The sseC gene is described in Medina et al., Infection and Immunity, 1999; 67(3): 1093-1099. The function of the encoded product is unknown.

The ssaB gene is described in Hensel, Molecular Microbiology, 2000; 36(5):1015-1023. The encoded product is a known substrate protein for Spi2, and interacts with normal endosomal trafficking in macrophages.

A second separate set of mutants comprise a first mutation that disrupts an arm gene. This mutation may be termed an “auxotrophic mutation” as the aro gene is essential in a biosynthetic pathway present in Salmonella, but not present in mammals. Therefore, the mutants cannot depend on metabolites found in the treated patient to circumvent the effect of the mutation. Suitable genes for the auxotrophic mutation, include aroA, aroC, aroD and aroE. In the preferred embodiment, aroC is disrupted.

The second mutation disrupts any of the clpP or sifA genes. ClpP is described above. The sifA gene is described in Stein et al., Mol. Microbiol., 1996; 20(1):151-164 and Beuzon et al., EMBO J., 2000; 19(13): 3235-3249. The sifA gene product is involved in the production in epithelial cells of lysosomal glycoprotein-containing structures.

The mutations may be introduced into the microorganism using any known technique. Preferably, the mutation is a deletion mutation, where disruption of the gene is caused by the excision of nucleic acids. Alternatively, mutations may be introduced by the insertion of nucleic acids or by point mutations. Methods for introducing the mutations into the specific regions will be apparent to the skilled person.

For example, gene deletions may be created by first amplifying the target gene plus flanking DNA using PCR and a high fidelity polymerase. The amplified product may then be cloned into a suitable cloning vector. PCR primers can be designed to delete the gene when used in inverse PCR, to generate an initial construct. The PCR primers may contain an Xbal site to introduce a new restriction site and thus provide a marker for the gene deletion. The deletion construct can then be transferred to a suicide vector for transfer to the Salmonella chromosome. This construct can be electroporated or conjugated into the desired strain, and recombinants containing the plasmid integrated into the chromosome at the homologous site (merodiploids), selected using an antibiotic resistance marker carried on the plasmid. The suicide vector may also contain the sacB gene that encodes the enzyme levan sucrase, which is toxic to most Gram-negative bacteria in the presence of sucrose. Sucrose selection may therefore be employed to isolate colonies where a second recombination event has occurred, resulting in loss of the plasmid from the chromosome. This second recombination event can result in two outcomes, re-generation of the wild-type allele or generation of a deletion mutant. Colonies containing the deletion mutation may then be identified by colony-PCR and the deletion confirmed by Southern blot analysis.

In addition to the two mutations, the Salmonella microorganism may also comprise heterologous antigens. The attenuated microorganism can therefore act as a delivery vehicle for administering antigens against other bacterial or viral infections. Antigens which are suitable for use in this way will be apparent to the skilled person and include:

Pathogenic E. coli antigens, i.e. ETEC

Hepatitis A, B and C antigens

Lime disease antigens

Vibrio cholera antigens

Helicobacter antigens

Herpes Simplex virus antigens

Human papilloma virus antigens

This system also has the potential to deliver therapeutic proteins, peptides or nucleic acids for the treatment of patients, e.g. patients infected with hepatitis. Cytokines are an example of suitable therapeutic proteins which may be delivered by the mutant microorganisms. Methods for the delivery of heterologous antigens or therapeutic proteins using the vaccine compositions will be apparent to the skilled person.

Vaccines made using the microorganisms of the invention have application to the treatment of infections in human patients and in the treatment of veterinary infections.

The double mutation provides an effective means to attenuate the microorganism to provide a safe vaccine candidate.

The vaccine compositions provide effective protection even in immuno-compromised patients, and importantly offer a low risk in developing spleen abscesses. Spleen abscesses have been identified using vaccines based on a single mutation, and therefore the present compositions may offer a substantial benefit to patients.

To formulate the vaccine compositions, the mutant microorganisms may be present in a composition together with any suitable pharmaceutically acceptable adjuvant, diluent or excipient. Suitable formulations will be apparent to the skilled person. The formulations may be developed for any suitable means of administration. Preferred administration is via the oral or intravenous routes and the vaccines are live attenuated Salmonella microorganisms. The number of microorganisms that are required to be present in the formulations can be determined and optimised by the skilled person. However, in general, a patient may be administered approximately 10⁷-10¹⁰ CFUs of the microorganism, preferably approximately 10⁸-10⁹ CFUs per single dosage unit.

1. A Salmonella microorganism having an attenuating mutation which disrupts the expression of a gene located within the Spi2 pathogenicity island, and a further mutation which disrupts the expression of any of the genes clpP, ompR, sifA, sseC and ssaB.

2. A Salmonella microorganism having an attenuating mutation which disrupts the expression of an aro gene, and a further mutation which disrupts the expression of any of the genes clpP and sifA.

3. A microorganism according to claim 2, wherein the aro gene is aroC.

4. A microorganism according to claim 1, wherein the Spi2 gene is ssaV, and the further mutation disrupts clpP, ompR, sifA or sseC.

5. A microorganism according to claim 1, wherein the Spi2 gene is ssaT, and the further mutation disrupts ssaB.

6. A microorganism according to any preceding claim, which further comprises a heterologous antigen or a therapeutic protein.

7. A microorganism according to claim 6, wherein the antigen is a hepatitis A, B or C antigen.

8. A microorganism according to any preceding claim, wherein the microorganism is Salmonella typhi Ty2.

9. A microorganism according to any preceding claim, for use in therapy.

10. A vaccine composition comprising a microorganism according to any of claims 1 to 8, an adjuvant and a physiologically acceptable diluent.

11. A composition according to claim 10, comprising from 10⁷-10¹⁰ CFUs of the microorganism per dosage unit.

12. A composition according to claim 11, comprising 10⁸-10⁹ CFUs of the microorganism per dosage unit.

13. Use of a microorganism as defined in any of claims 1 to 8, in the manufacture of a medicament for the treatment of systemic bacterial infection.

14. Use according to claim 13, wherein the infection is typhoid.
